(19) United States
(12) Patent Application Publication
(10) Pub. No.: US 2005/0113781 A1
(43) Pub. Date: May 26, 2005

(54) Device for urinary incontinence

(76) Inventors: Sylviane Forgeot, Paris (FR); Veronique Aubert, Paris (FR)

Correspondence Address: Richard P. Gilly;Wolf, Block, Schorr & Solis-Cohen LLP, 22nd Floor, 1650 Arch Street, Philadelphia, PA, 19103, US

(21) Appl. No.: 10/673,979

(22) Filed: Sep. 29, 2003

(30) Foreign Application Priority Data
Jul. 4, 2003 (FR) 03 08220

(51) Int. Cl. A61F 13/15

(52) U.S. Cl. 604/385.18

(57) ABSTRACT

The purpose of the invention is an impermeable device for urinary incontinence with an approximately cylindrical shape, comprising cotton fibres covered by a composition. This device is characterized in that the cotton fibres are compacted, in that the fibres forming the outer surface of the device are smooth over their entire length, and in that the composition partly or completely covering the cotton fibres is a lubricating composition, making the device impermeable and having a therapeutic and/or moisturizing effect.

This invention relates to a device for urinary incontinence.

Some women, particularly who have given birth to children or women after their menopause, are subject to involuntary urine leaks. For example, this type of leak may be related to an effort made when coughing, laughing, during stress, when practicing some sports or without any obvious reasons.

This is then a major handicap for these women in their daily life.

One of several techniques known at the moment consists of a surgical operation that consists of fitting a wire or a strip under the lower part of the urethra to support it, and thus preventing this uncontrolled flow of urine.

Unfortunately this technique that is particularly difficult to use, also has the disadvantage that it does not always correctly and completely solve this problem of female incontinence. This type of technique is usually used in the case of severe incontinence, but it also creates the problem that it has to be repeated because the results obtained are unsatisfactory, with all the disadvantages related to hospitalisation.

Another possible technique known in the past for treat female urinary incontinence consists of applying electrical pulses at the entrance to the vagina.

But this type of technique is expensive and difficult to apply, and does not necessarily give all the expected results.

Furthermore, oral absorption of some medicine designed to reduce contractions of the bladder causes particularly unpleasant secondary effects, such as dryness in the mouth.

Some of the simplest known techniques in practice are described in documents WO00/37013 and WO02/26160. Each of these documents describes an impermeable device for female urinary incontinence, composed of two distinct elements forming the inner core of the device and its outer envelope. The inner core composed of a physically strong member is designed to provide mechanical support to the urethra, while the outer envelope is designed to come into direct contact with the vaginal mucous membranes.

Despite its relatively simple composition, this type of device requires the assembly of different elements. This document does not describe the presence of a lubricating cream, which in particular would make vaginal mucous membranes more supple to prevent them from being irritated when the device is put into place and removed.

Moreover, document WO00/67662 describes a device for urinary incontinence comprising at least one curved, supple support means designed for mechanical support of the urethra, and a means of absorbing urine leaks.

However, absorption of urine leaks by this type of device still causes the presence of unpleasant smells, more or less pronounced and particularly annoying for the user and possibly for her immediate surroundings.

Thus, there remains a need for a rigid device for female urinary incontinence, with a much simpler composition than devices known in the past, that provides perfect mechanical support of the urethra without any major deformations during the day while it is being worn, that is easy to insert into and remove from the vagina, that is comfortable to wear all day, and that does not absorb any urine leaks.

Therefore, the purpose of the invention is an impermeable device for urinary incontinence with an approximately cylindrical shape, comprising cotton fibres covered by a composition. The device is characterized in that the fibres forming the outer surface of the device are smooth over their entire length, and in that the composition partly or completely covering the cotton fibres is a lubricating composition, making the device impermeable and having a therapeutic and/or moisturising effect.

The device according to the invention has the advantage that its composition is significantly simpler than similar devices existing at the moment, that it is leak tight to any liquid originating from the organism, that it has a relatively low cost and in addition to mechanical support, it facilitates care of the mucous membranes inside the inner female tractus due to the presence of compounds with a therapeutic effect.

The composition with a therapeutic effect that is preferably present on the device comprises an agent chosen from among estrogens, anti-microbial agents, anti-inflammatory agents, anti-viral agents and mixes of these substances.

The composition with a moisturising effect may be chosen from among moisturising creams, but it is not the same as moisturising gels.

To simplify removal, the device according to the invention may include a means of removal (for example a string) that may be fixed to the inside and/or the outside of the cotton fibres. In the same way as for its removal, the device according to the invention may include an applicator, preferably made of plastic, to facilitate placement in the organism.

We will now describe the invention with reference to the following examples based on the figures attached, and in which:

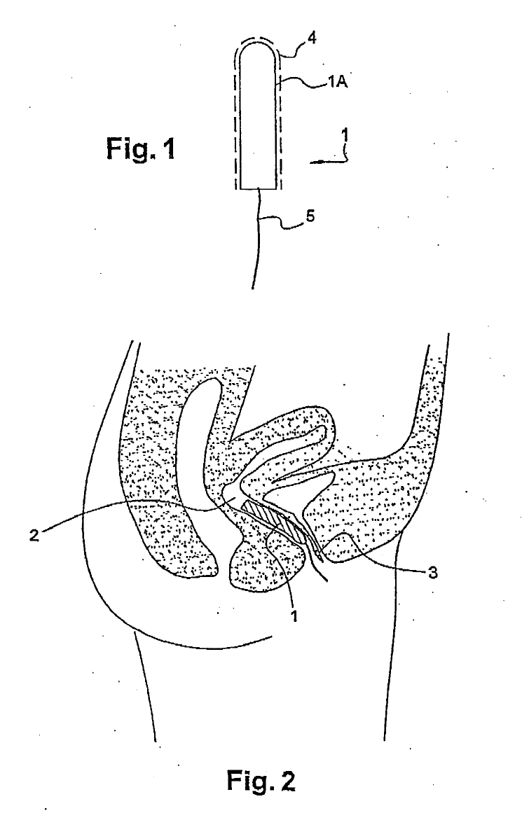

FIG. 1 shows a diagrammatic sectional view of the device according to the invention,

FIG. 2 shows a diagrammatic sectional view of the female pelvis including the device according to the invention.

As can be seen in FIG. 1, the device according to the invention with general reference 1 is approximately cylindrically shaped. It is composed of a set of very dense compacted cotton fibres that are sufficiently rigid to penetrate into the entrance to the vagina without being deformed in any way during penetration, or while providing daily mechanical support. The length of the device 1 may be between 4 and 6 cm, and preferably 5 cm, and its diameter may be between 1 and 1.5 cm, and preferably 1 cm.

The device according to the invention may be available in different sizes. The smallest (about 5 cm) may be provided with less lubricating composition than the larger device, to achieve a better fit to the organism of different users.

Fibres forming the outer part 1A of the device are perfectly smooth and there are no grooves between them, to maintain a perfect seal from urine leaks.

As can be seen in FIG. 2, and due to its composition based on cotton fibres only, once the device 1 according to the invention has been placed in the vagina 2, it easily and quickly matches the volume in which it is placed, partly due to body heat.

Thus, due to the presence of device. 1, the urethra 3 is mechanically moved upwards the top of the pelvis. As a result of this action to lift the urethra upwards, the device 1 according to the invention closes the outlet from the urethra, and therefore prevents any urine leaks to the outside.

The presence of a composition 4 that coats all or some of the device 1 makes it impermeable so that it will not absorb any urine. This means that it remains clean and that it does not smell bad throughout the day while it is being worn.

Furthermore, apart from its impermeabilising effect, the composition 4 can give perfect leak tightness at the closure of the urethra.

Finally, the presence of this composition 4 on the outer fibres of the device 1 facilitates penetration or withdrawal of the device into the vagina, even several times during the day without any risk of irritation and/or pain as a result of these operations.

The device 1 according to the invention will preferably be removed every time that the user wants to urinate to avoid unnecessarily soiling the device, and consequently will have to be put back into place after she has finished urinating.

The device according to the invention is preferably placed in the vagina in the morning during the morning toilet, and is removed in the evening and left out before going to bed. The advantage of this type of device is that it provides daily mechanical support to the urethra, with the use of a single device according to the invention throughout the day, since it is not soiled during the day.

Wearing the device according to the invention every day does not in any way disturb the urine cycle.

The presence of the composition 4 on the perfectly smooth outer fibres provides a means of combining perfect leak tightness of the device to urine leaks, and a perfect fit of the device onto the vaginal mucous membranes.

For women who still have their monthly menstruation, the device 1 according to the invention cannot be used during the few days of the menstruation period because it does not absorb any liquid. It can then be replaced by conventional protection known for this purpose.

The device according to the invention may also include a means 5 of withdrawing it, such a string to make it easy to pull out of the organism. The removal device may be fixed by any known attachment means in and/or around the compacted cotton fibres so that it does not become detached during the day as a result of regularly removing the device from the organism to urinate.

The device according to the invention may be presented in an individual case like those usually used, made of plastic or a metal such as aluminium. An applicator to assist placement in the organism may also be included in the same packaging, with different presentation variants.

1. Impermeable device for urinary incontinence with an approximately cylindrical shape, comprising cotton fibres covered by a composition, characterized in that the said fibres are compacted, in that the fibres forming the outer surface of the device are smooth over their entire length, and in that the composition partly or completely covering the cotton fibres is a lubricating composition, making the device impermeable and having a therapeutic and/or moisturising effect

2. Device according to claim 1, characterized in that the composition with a therapeutic effect comprises an agent chosen from among estrogenic agents, anti-microbial agents, anti-inflammatory agents, anti-viral agents and mixes of these substances

3. Device according to claim 1, characterized in that the composition with a moisturising effect is chosen from among moisturising creams.

4. Device according to claim 1, characterized in that it also comprises a removal means, the said means being fixed to the inside and/or the outside of the cotton fibres.

5. Device according to claim 1, characterized in that it also comprises an applicator to facilitate placement in the organism.

6. Device according to claim 1, characterized in that its length is between about 4 and 6 cm, and preferably 5 cm.

7. Device according to claim 1, characterized in that its diameter is between 1 and 1.5 cm, and preferably 1 cm.
